(19) United States
(12) Patent Application Publication
(10) Pub. No.: US 2021/0259885 A1
(43) Pub. Date: Aug. 26, 2021

(54) Device For Ablation Processing Of Ophthalmological Implantation Material

(71) Applicant: Ziemer Ophthalmic Systems AG, Port (CH)

(72) Inventor: Christian Rathjen, Bremen (DE)

(21) Appl. No.: 17/186,652

(22) Filed: Feb. 26, 2021

(30) Foreign Application Priority Data
Feb. 26, 2020 (CH) 00231/20

(51) Int. Cl. A61F 9/008

(52) U.S. Cl. CPC A61F 9/00814; A61F 9/00812; A61F 2009/00872; A61F 2009/0087; A61F 2009/00897

(57) ABSTRACT

A device for ablation processing of ophthalmological implantation material, which is formed by water-containing base material, comprises a laser source, which is configured to generate a pulsed laser beam having a processing wavelength in the ultraviolet wavelength range, wherein the processing wavelength is greater than 193 nm and causes a higher absorptance of the laser beam in the base material of the implantation material than the absorptance of the laser beam in the water of the implantation material is described.

### CROSS-REFERENCE TO RELATED APPLICATION

The present application claims priority to and the benefit of Switzerland Patent Application 00231/20 filed Feb. 26, 2020, the content of which is incorporated by reference in its entirety herein.

### FIELD OF THE TECHNOLOGY

The present disclosure relates to a device for ablation processing of ophthalmological implantation material. The present disclosure relates in particular to a device for ablation processing of ophthalmological implantation material, which is formed by water-containing base material.

### BACKGROUND

Laser ablation methods and laser devices suitable for this purpose, which cause material removal via absorption of laser energy at the surface, are known. Greatly varying methods, laser wavelengths, and pulse durations are used for this purpose. FIG. 4 is based on a publication by Michael Kaschke, Karl-Heinz Donnerhacke, and Michael Stefan Rill, “Optical Devices in Ophthalmology and Optometry: Technology, Design Principles, and Clinical Applications”, 22 Jan. 2014, Wiley-VCH Verlag GmbH & Co. KGaA, and illustrates the delimitation of ablation methods in relation to other laser material processing methods. As is apparent from FIG. 4, photoablation methods are distinguished by a parameter range PA, which works with pulsed laser beams having intensities I in the range of approximately 10⁷ W/cm² to approximately 10¹⁰ W/cm² and action times or pulse durations D of approximately 10⁻⁹ seconds to approximately 10⁻⁶ seconds. As is moreover apparent in FIG. 4, this corresponds to radiation energies E in the range of approximately 10⁻¹ J/cm² to approximately 10³ J/cm². In medical technology, ablation methods are executed using wavelengths in the ultraviolet (UV) and in the infrared (IR) range, because there is an increased absorption in water in these wavelength ranges.

Ablation methods are used for processing various materials, for example for processing hard materials such as teeth or diamonds. For the ablation processing of ophthalmological tissue material in refractive surgery, excimer lasers are used, since their short wavelengths are strongly absorbed by water and proteins and deep penetration into the eye tissue and inadvertent tissue damage linked thereto do not occur. The wavelengths (193 nm) of ArF (argon fluoride) excimer lasers are absorbed many times (approximately 20 times) more strongly by water than wavelengths which are greater than 200 nm. Accompanying this, the humidity in the tissue to be processed and a possible moisture film on the tissue has a comparatively substantially stronger influence on ablation removal in the case of processing using ArF excimer lasers than in the case of longer wavelengths. Therefore, in refractive surgery the cornea is dried before the ablation, in particular by swabbing, and the humidity in the operating room is set in a defined manner in order to avoid excessively strong moisture of the cornea and increased absorption in the water accompanying this.

### SUMMARY

It is an object of the present disclosure to propose a device for ablation processing of ophthalmological implantation material, which does not have at least some disadvantages of the known systems.

According to the present disclosure, these goals are achieved by the features of the independent claim. Further advantageous illustrative examples are additionally disclosed in the dependent claims and the description.

The above-mentioned goals are in particular achieved by the present disclosure in that a device is provided for ablation processing of ophthalmological implantation material formed by water-containing base material, which comprises a laser source which is configured to generate a pulsed laser beam having a processing wavelength in the ultraviolet wavelength range, wherein the processing wavelength is greater than 193 nm and causes a higher absorptance of the laser beam in the base material of the implantation material than the absorptance of the laser beam in the water of the implantation material. The device additionally comprises a projection lens, which is configured to radiate the pulsed laser beam onto a surface of the implantation material and, in a processing region, to trigger an interaction with the implantation material for the ablation of the implantation material using laser pulses of the laser beam, which laser pulses have a combination of pulse duration and intensity effectuating photoablation. The device furthermore comprises a scanner device, which is configured to execute a movement of the processing region for ablation processing according to a processing pattern.

In one illustrative example variant, the laser source is configured to generate the pulsed laser beam having a processing wavelength in a wavelength range which is delimited in the lower wavelength range by a maximum absorptance to be achieved of 10⁻²/cm of the laser beam in the water of the implantation material and which is delimited in the higher wavelength range by a minimum absorptance to be achieved of 10⁰/cm of the laser beam in the base material of the implantation material.

In one illustrative example variant, the laser source is configured to generate the pulsed laser beam at a processing wavelength in the ultraviolet wavelength range of greater than 200 nm.

In one illustrative example variant, the laser source is configured to generate the pulsed laser beam at a processing wavelength in a wavelength range from 200 nm to 250 nm.

In one illustrative example variant, the pulse duration of the laser pulses is in a pulse duration range of 10⁻¹⁰ seconds to 10⁻⁵ seconds, in particular in a pulse duration range of 10⁻⁹ seconds to 10⁻⁶ seconds.

In one illustrative example variant, the intensity of the laser pulses is in an intensity range of 10⁶ W/cm² to 10¹¹ W/cm², in particular in an intensity range of 10⁷ W/cm² to 10¹⁰ W/cm².

In one illustrative example variant, the laser source and the projection lens are configured to radiate the pulsed laser beam with a fluence in the fluence range of 10⁶ W/cm² and 10¹⁰ W/cm² onto the surface of the implantation material.

In one illustrative example variant, the device comprises an air humidifier, an humidity sensor, and a control unit interconnected with the air humidifier and the humidity sensor. The control unit comprises an electronic circuit which is configured to control the air humidifier as a function of an humidity value measured by the humidity sensor in a surroundings region adjacent to the implantation material in such a way that a predetermined minimum humidity value is maintained.

In one illustrative example variant, the electronic circuit is configured to control the air humidifier in such a way that a minimum humidity value of 90% relative humidity is maintained, in particular a minimum humidity value of 95% relative humidity.

In one illustrative example variant, the scanner device is configured to execute the movement of the processing region for the ablation processing according to a processing pattern for generating a lenticular surface.

In one illustrative example variant, the scanner device comprises at least one movable mirror, which is configured to deflect the pulsed laser beam for the movement of the processing region according to the processing pattern.

In one illustrative example variant, the scanner device is arranged downstream of the projection lens.

In one illustrative example variant, the scanner device comprises at least one drive, which is configured to displace the projection lens in order to execute the movement of the processing region according to the processing pattern.

In one illustrative example variant, the scanner device comprises at least one drive, which is configured to displace a material carrier, on which the implantation material is applied, in order to execute the movement of the processing region according to the processing pattern.

### BRIEF DESCRIPTION OF THE DRAWINGS

An illustrative example of the present disclosure is described hereinafter on the basis of an example. The example of the illustrative example is illustrated by the following appended figures:

FIG. 1 schematically shows a cross section of a block diagram having illustrative example variants of a device for ablation processing of ophthalmological implantation material.

FIG. 2 schematically shows a cross section of a block diagram having further illustrative example variants of a device for ablation processing of ophthalmological implantation material.

FIG. 3 shows a graph which illustrates the absorption of laser light in water and in protein forming the base material of the cornea as a function of the wavelength of the laser beam.

FIG. 4 shows a graph which illustrates the parameter ranges of various material processing methods by means of lasers.

### DETAILED DESCRIPTION

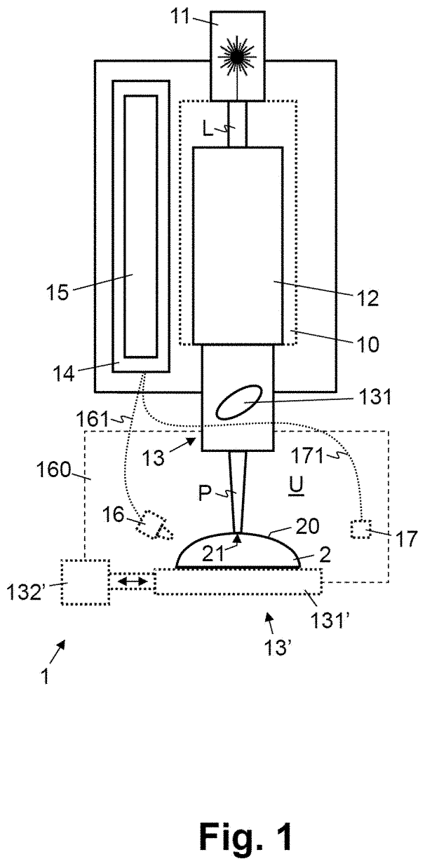

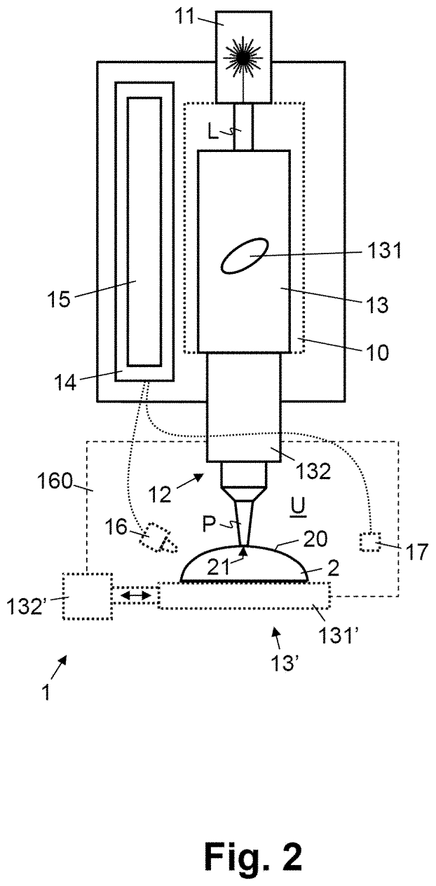

In each of FIGS. 1 and 2, the reference sign 1 refers to a device for ablation processing, in particular a device for ablation processing of ophthalmological implantation material 2, in particular of water-containing ophthalmological implantation material 2. The ophthalmological implantation material 2 thus comprises base material and water. The ophthalmological implantation material 2 comprises natural donor tissue, for example human corneal tissue (cornea) having protein(s) as the base material, or synthetic tissue, for example hydrogels, which comprise polymers containing water.

As schematically shown in FIGS. 1 and 2, the device 1 comprises a laser source 11, a projection lens 12, a scanner device 13, 13′, and a control unit 14 having an electronic circuit 15. The control unit 14 or the electronic circuit 15, respectively, is interconnected via signal and/or control lines to the laser source 11, the projection lens 12, and the scanner device 13, 13′ for their control.

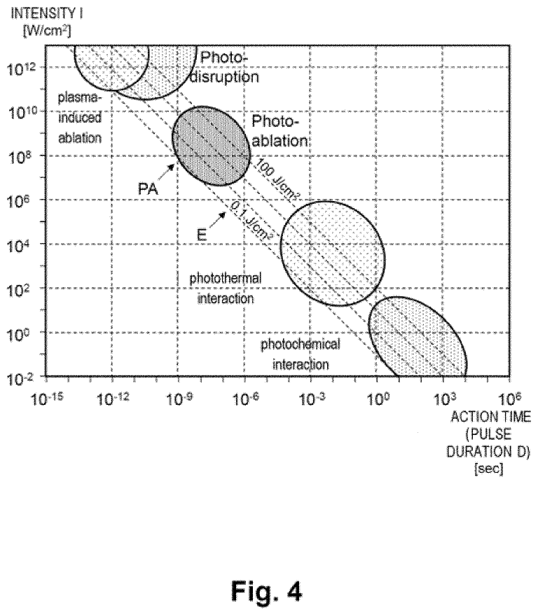

The laser source 11 is configured to generate a pulsed laser beam L having a processing wavelength λ in the ultraviolet wavelength range, as explained and defined in greater detail hereinafter. The projection lens 12 is configured to radiate the pulsed laser beam L onto a surface 20 of the implantation material 2 and to trigger an interaction with the implantation material 2 for ablation of the implantation material 2 in a processing region 21 using laser pulses P of the laser beam L. For this purpose, the laser pulses P generated by the laser source 11 and radiated by the projection lens 12 onto the surface 20 of the implantation material 2 have a combination of pulse duration D and intensity I in a parameter range PA, which effectuate photoablation (see FIG. 4).

As is additionally schematically shown in FIGS. 1 and 2, the device 1, in one illustrative example variant, comprises an humidity sensor 17 and an air humidifier 16, which are attached, for example, in a closed humidity chamber 160. The humidity sensor 17 and the air humidifier 16 are interconnected via a signal line 171 or via a control line 161 to the control unit 14 or to its electronic circuit 15, respectively. The electronic circuit 15 is embodied as a programmed processor, as an application-specific integrated circuit (ASIC), or as another electronic logic unit.

The electronic circuit 15 ascertains, via the signal line 171, the relative humidity measured by the humidity sensor 17 in the surroundings region U of the implantation material 2 to be processed. The electronic circuit 15 is configured to control the air humidifier 16 as a function of the measured humidity value in such a way that a predetermined minimum humidity value is maintained. A water tank and/or a water conduit for supplying water to the air humidifier 16 is not shown in FIGS. 1 and 2. The minimum humidity value is, for example, at least 90% relative humidity, in particular 95% relative humidity. The optional humidity chamber 160 schematically shown in FIGS. 1 and 2 simplifies and increases the accuracy of the relative humidity to be maintained.

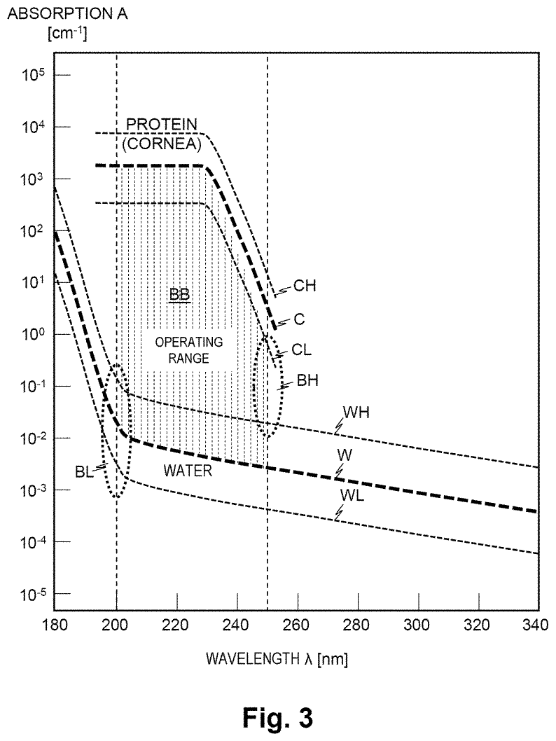

The laser source 11 is configured to generate a pulsed laser beam L having a wavelength λ in the ultraviolet wavelength range, wherein the wavelength λ is greater than 193 nm. The laser source 11 is moreover configured to generate the pulsed laser beam L having a wavelength λ in a wavelength range, in which the wavelength λ causes a higher absorptance A of the pulsed laser beam L in the base material of the implantation material 2 than in the water of the implantation material 2, for example in an operating range BB according to FIG. 3. The laser source 11 is thus configured to generate the pulsed laser beam L having a processing wavelength λ, which is greater than 193 nm, i.e., greater than the wavelength of known ArF excimer lasers, on the one hand, and has a higher absorptance A in the base material of the implantation material 2 than in water, on the other hand.

This relationship of wavelength λ and absorptance A in the base material of the implantation material 2, on the one hand, and in water, on the other hand, is shown in FIG. 3. FIG. 3 illustrates, as a function of the wavelength λ of the laser beam L, the absorptance A of laser light in water and in protein, as an example of base material of the cornea. The profile of the absorptance A as a function of the wavelength λ is shown for water using the curves WH, W, and WL. The wavelength-dependent absorption curves WH and WL for water illustrate a value range having a high or low absorption rate, respectively, of light in water. The wavelength-dependent absorption curve W for water corresponds to a mean absorptance of light in water. The different wavelength-dependent absorption curves WH, W, and WL are defined, on the one hand, by different degrees of purity of the water and different measurement conditions. The profile of the absorptance A as a function of the wavelength λ is shown for protein as the base material of the cornea using the curves CH, C, and CL. The wavelength-dependent absorption curves CH and CL for protein (cornea) illustrate a value range having a high or low absorption rate, respectively, of light in the protein (cornea). The wavelength-dependent absorption curve C for protein (cornea) corresponds to a mean absorptance of light in the protein (cornea). The different wavelength-dependent absorption curves CH, C, and CL are defined in particular by different measurement conditions.

The reference sign BB in FIG. 3 identifies the operating range for the laser source 11 of the device 1. As illustrated in FIG. 3, the operating range BB is determined, on the one hand, by the profile of the wavelength-dependent absorption curves WH, W, and WL for water and, on the other hand, by the wavelength-dependent absorption curves CH, C, and CL for protein (cornea), for example by the mean wavelength-dependent absorption curve W for water and the mean wavelength-dependent absorption curve C for protein (cornea). The operating range BB for the laser source 11 is determined so that the absorption A of the pulsed laser beam L in the protein (cornea) as a function of the wavelength λ is always greater than in water. In the lower wavelength range, at approximately 200 nm, the operating range BB is delimited by the steep increase of the absorptance A in the water for wavelengths below this (in the range identified by BL). In the upper wavelength range, the operating range BB is delimited by the drop of the absorptance A in the protein (cornea) at approximately 250 nm. The absorptance CL in the protein (cornea) approaches the absorptance in the water WH there in such a way that in the extreme case (in the range identified by BH), when the wavelength-dependent absorption curve WH having a high absorptance in water and the wavelength-dependent absorption curve CL having a low absorptance CL in the protein (cornea) are taken into consideration, the difference in the absorption rates in water WH and in the protein (cornea) CL falls to a factor less than 10² and approaches the factor 10¹.

FIG. 1 schematically shows an exemplary illustrative example, in which a scanner device 13 having one or more movable mirrors 131 for deflecting the pulsed laser beam L is interconnected downstream of the projection lens 12. In this case, the pulsed laser beam L is prepared (focusing/converging) by the projection lens 12 for the irradiation of the surface 20 in a processing region 21 of the implantation material 2 to be treated. The processing region 21 is moved by the scanner device 13 by deflection of the pulsed laser beam L by means of one or more movable mirrors 131 according to a processing pattern, for example to generate a lenticular surface of a lenticule to be produced from the implantation material 2.

FIG. 2 schematically shows an exemplary illustrative example in which the scanner device 13 having one or more movable mirrors 131 is interconnected upstream of the projection lens 12.

In further illustrative example variants, the scanner device 13 comprises one or more drives 132 for displacing the projection lens 12 in order to execute the movement of the processing region 21 according to the processing pattern.

A further alternative illustrative example variant of the scanner device 13′ is illustrated schematically both in FIG. 1 and also in FIG. 2. The scanner device 13′ comprises one or more drives 132′, which are configured to displace a material carrier 131′, on which the implantation material 2 to be processed is applied, in order to execute the movement of the processing region 21 according to the processing pattern.

What is claimed is:

1. A device for ablation processing of ophthalmological implantation material, which is formed by water-containing base material, comprising:
a laser source configured to generate a pulsed laser beam having a processing wavelength in the ultraviolet wavelength range, wherein the processing wavelength is greater than 193 nm and causes a higher absorptance of the pulsed laser beam in the base material of the implantation material than absorptance of the laser beam in the water of the implantation material;
a projection lens configured to radiate the pulsed laser beam onto a surface of the implantation material, and, in a processing region, to trigger an interaction with the implantation material for ablation of the implantation material using laser pulses of the laser beam, wherein the laser pulses have a combination of pulse duration and intensity causing photoablation; and
a scanner device configured to execute a movement of the processing region for the ablation processing according to a processing pattern.

2. The device of claim 1, wherein the processing wavelength is delimited in a lower wavelength range by a maximum absorptance of 10⁻²/cm of the laser beam in the water of the implantation material and is delimited in a higher wavelength range by a minimal absorptance of 10⁰/cm of the laser beam in the base material of the implantation material.

3. The device of claim 1, wherein the processing wavelength is greater than 200 nm.

4. The device of claim 1, wherein the processing wavelength is in a range of 200 nm to 250 nm.

5. The device of claim 1, wherein the pulse duration is in a pulse duration range of 10⁻⁹ seconds to 10⁻⁶ seconds.

6. The device of claim 1, wherein the intensity is in an intensity range of 10⁷ W/cm² to 10¹⁰ W/cm².

7. The device of claim 1, wherein the laser source and the projection lens are further configured to radiate the pulsed laser beam with a fluence in a fluence range of 10⁶ W/cm² and 10¹⁰ W/cm² onto the surface of the implantation material.

8. The device of claim 1, further comprising:
an air humidifier;
an humidity sensor; and
a control unit, interconnected to the air humidifier and the humidity sensor, comprising an electronic circuit configured to control the air humidifier as a function of an humidity value measured by the humidity sensor in a surroundings region adjacent to the implantation material in such a way that a predetermined minimum humidity value is maintained.

9. The device of claim 8, wherein the electronic circuit further is configured to control the air humidifier in such a way that a minimum humidity value of 95% relative humidity is maintained.

10. The device of claim 1, wherein the scanner device further is configured to execute the movement of the processing region for ablation processing according to the processing pattern to generate a lenticular surface.

11. The device of claim 1, wherein the scanner device comprises at least one movable mirror configured to deflect the pulsed laser beam for the movement of the processing region according to the processing pattern.

12. The device of claim 11, wherein the scanner device is arranged downstream of the projection lens.

13. The device of claim 1, wherein the scanner device comprises at least one drive configured to displace the projection lens in order to execute the movement of the processing region according to the processing pattern.

14. The device of claim 1, wherein the scanner device comprises at least one drive configured to displace a material carrier, on which the implantation material is applied, in order to execute the movement of the processing region according to the processing pattern.

15. A method for ablation processing of ophthalmological implantation material, which is formed by water-containing base material comprising:
generating a pulsed laser beam having a processing wavelength in the ultraviolet wavelength range, wherein the processing wavelength is greater than 193 nm and causes a higher absorptance of the pulsed laser beam in the base material of the implantation material than absorptance of the laser beam in the water of the implantation material;
radiating the pulsed laser beam onto a surface of the implantation material;
triggering, in a processing regions, an interaction with the implantation material for ablation of the implantation material using laser pulses of the laser beam, wherein the laser pulses have a combination of pulse duration and intensity causing photoablation; and
executing a movement of the processing region for the ablation processing according to a processing pattern.

16. The method of claim 15, further comprising controlling an air humidifier as a function of a humidity value measured by a humidity sensor in a surroundings region adjacent to the implantation material in such a way that a predetermined minimum humidity value is maintained.

17. The method of claim 16, controlling the air humidifier in such a way that a minimum humidity value of 95% relative humidity is maintained.

18. The method of claim 15, further comprising executing the movement of the processing region for ablation processing according to the processing pattern to generate a lenticular surface.

19. The method of claim 15, further comprising deflecting the pulsed laser beam for the movement of the processing region according to the processing pattern.

20. The method of claim 15, further comprising displacing a material carrier, on which the implantation material is applied, in order to execute the movement of the processing region according to the processing pattern.
